(19) United States
(12) Patent Application Publication
(10) Pub. No.: US 2008/0200685 A1
(43) Pub. Date: Aug. 21, 2008

(54) Method for Preforming Oxidation Catalysts

(75) Inventors: Samuel Neto, Dresden (DE); Frank Rosowski, Mannheim (DE); Sebastian Storck, Mannheim (DE); Jurgen Zuhlke, Speyer (DE); Hans-Martin Allmann, Neunkirchen (DE); Thomas Lautensack, Birkenau (DE); Rainer Steeg, Ludwigshafen (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/916,731

(22) PCT Filed: May 31, 2006
(86) PCT No.: PCT/EP2006/062787

§ 371 Date: Dec. 6, 2007

(30) Foreign Application Priority Data
Jun. 7, 2005 (DE) 10 2005 026 360.7

(51) Int. Cl. B01J 37/08; C07C 51/16; C07D 307/34; C07D 307/89; C07D 211/00

(52) U.S. Cl. 546/318; 502/439; 562/408; 549/256; 549/248

(57) ABSTRACT

Processes comprising providing a catalyst precursor, and heating the catalyst precursor to a temperature of at least 350° C. in an atmosphere comprising air, wherein air is fed into the atmosphere at a rate of 0.05 to 4.0 standard m³/h, and wherein the catalyst precursor is activated at a temperature of at least 350° C. for more than 9 hours are described along with catalysts formed thereby and uses for such catalysts.

The invention relates to a process for preactivating oxidation catalysts, wherein the catalyst precursor is heated to a temperature of at least 350° C. in an atmosphere comprising air and having an amount of air fed in of from 0.05 to 4.0 standard m³/h and the catalyst precursor is activated at least 350° C. for at least 9 hours.

Coated catalysts in which the catalytically active composition has been applied in the form of a shell to an inert support material such as steatite have been found to be useful as oxidation catalysts. The catalytically active constituent of the catalytically active composition of these coated catalysts comprises, for example, titanium dioxide (in the form of its anatase modification) and vanadium pentoxide. Furthermore, small amounts of many other oxidic compounds which act as promoters to influence the activity and selectivity of the catalyst can be comprised in the catalytically active composition.

To produce such coated catalysts, a solution or suspension of the constituents of the active composition and/or precursor compounds thereof in an aqueous medium and/or an organic solvent is sprayed onto the support material at elevated temperature until the desired proportion by weight of active composition in the catalyst has been achieved.

To improve the quality of the coating, it has become industrial practice to add organic binders, preferably copolymers, advantageously in the form of an aqueous dispersion, of vinyl acetate-vinyl laurate, vinyl acetate-acrylate, styrene-acrylate, vinyl acetate-ethylene or acrylic acid-maleic acid, to the suspension. Coating is generally carried out at temperatures of from room temperature to 200° C. The addition of binder also has the advantage that the active composition adheres well to the support, so that transport and charging of the catalyst are made easier.

The preactivation is usually carried out at temperatures from >200 to 500° C. During this thermal treatment the binder is driven off from the applied layer by thermal decomposition and/or combustion. The thermal treatment/preactivation is usually carried out in situ in the oxidation reactor.

DE-A 25 50 686 describes a process for producing catalysts for oxidation reactions in the gas phase. As binders which are added to the coating solution, mention is made of urea compounds such as urea, thiourea, cyanamide compounds or dicyanamides. It is stated that the duration of the activating treatment is not critical, but the time should be a minimum of 5 hours. In the example, the coated support is heated uniformly from 280 to 400° C. in a stream of air and maintained at this temperature for 6 hours.

U.S. Pat. No. 4,489,204 discloses a process for preparing phthalic anhydride using ring-shaped support material. In Example 1, it is stated that the catalyst is heated to 300° C. using an amount of air of 0.5 standard m³/h and preactivation is continued by heating the catalyst at a heating rate of 10° C./h to 390° C., with the second heating phase having a duration of 9 hours.

DE-A 103 35 346 discloses catalysts for gas-phase oxidations which comprise an inert support and a catalytically active composition comprising transition metal oxides applied thereto. As binder, mention is made of a copolymer of an α-olefin and a vinyl C₂-C₄ carboxylate whose vinyl C₂-C₄-carboxylate content is at least 62 mol %. It is stated that the binder is driven off from the applied layer by thermal decomposition and/or combustion by thermal treatment of the catalyst at temperatures of from >200 to 500° C.

EP-A 0 744 214 and DE-A 197 17 344 describe a supported catalyst and a process for producing catalysts in which a mixture of oxides is milled in the presence of water and subsequently applied to support bodies. Organic binders mentioned are vinyl acetate-vinyl laurate, vinyl acetate-acrylate, styrene-acrylate, vinyl acetate-maleate and vinyl acetate-ethylene. It is stated that the binder burns out quantitatively within a short time in the stream of air after the catalyst is introduced into the reactor.

U.S. Pat. No. 4,397,768 describes a catalyst for the preparation of phthalic anhydride. The active composition is applied to an inert support with the aid of organic binders such as vinyl acetate-vinyl laurate, vinyl acetate-acrylate, styrene-acrylate, vinyl acetate-maleate or vinyl acetate-ethylene. To burn out the binder, the catalysts are heated in the reactor to 380° C. using an amount of air fed in of 1 standard m³/h.

DE-A 198 24 532 discloses a binder for producing coated catalysts, which comprises a polymer of ethylenically unsaturated acid anhydrides and an alkanolamine having at least 2 OH groups, not more than 2 nitrogen atoms and not more than 8 carbon atoms. To test whether odorous or environmentally unfriendly substances are liberated on burning off the added binder, the catalyst was heated from 30° C. to 610° C. at a heating rate of 5° C./min while passing air through it.

It was an object of the present invention to provide an improved process for preactivating oxidation catalysts. In particular, the burnout of the binders used was to be optimized. Furthermore, the formation of carbon deposits was to be minimized and the start-up behavior of the catalysts optimized by means of an improved burnout process. Optimization of the start-up behavior can, for example, be achieved by a pronounced hot spot being formed in the first catalyst zone on starting up the reactor.

We have accordingly found a process for preactivating oxidation catalysts, wherein the catalyst precursor is heated to a temperature of at least 350° C. in an atmosphere comprising air and having an amount of air fed in of from 0.05 to 5.0 standard m³/h and the catalyst precursor is activated at least 350° C. for at least 9 hours.

The term “air” as used for the purposes of the present invention refers to a gas or a gas mixture which consists essentially of nitrogen, preferably having nitrogen contents of greater than 75% by volume, and oxygen, preferably having oxygen contents of greater than 15% by volume. Depending on the source from which the air comes, its composition can fluctuate within limits with which those skilled in the art are familiar. Ambient air is advantageously used as air source.

The catalyst precursor is advantageously heated to at least 370° C., preferably to 390-470° C. The temperature should preferably not exceed a value of 500° C.

After the desired temperature has been reached, the catalyst precursor is advantageously activated for at least 9 hours at this temperature, i.e. at at least 350° C., advantageously at least 370° C. and in particular from 390 to 470° C. The catalyst precursor is advantageously activated at the temperature specified for at least 12 hours, preferably for at least 15 hours, in particular for at least 24 hours.

The catalyst precursor is advantageously heated at a heating rate of from 3 to 12° C./h, preferably at a heating rate of from 5 to 10° C./h. The heating phase consequently has a duration of preferably from 25 to 120 hours, advantageously from 40 to 70 hours.

The amount of air used during heating is advantageously from 0.05 to 5.0 standard m³/h. The air can, if appropriate, be diluted with an inert gas. For example, the air is diluted in a ratio of air to inert gas of from 1:0.1 to 1:1, preferably in a ratio of from 1:0.1 to 1:0.2. Inert gases which can be used are all those known to those skilled in the art, for example nitrogen, carbon dioxide, argon and/or helium.

The heating phase can, if appropriate, be divided into a plurality of substeps, advantageously from two to ten substeps.

For example, the heating phase is divided into three substeps:

In a first heating stage, the catalyst precursor is heated at low temperatures from about room temperature to 80-120° C. using a small amount of air of advantageously from 0.05 to 3 standard m³/h, preferably from 0.1 to 1 standard m³/h;
in a second heating stage, the catalyst precursor is heated at intermediate temperatures from about 80-120° C. to 250-290° C. using an intermediate amount of air of advantageously from 1 to 4.5 standard m³/h, in particular from 2 to 4 standard m³/h; and
in a third heating stage, the catalyst precursor is heated at high temperatures from about 250-290° C. to 350-470° C. using a small amount of air of advantageously from 0.05 to 2.5 standard m³/h, in particular from 0.05 to 1.5 standard m³/h.

If appropriate, hold zones can be present after the individual stages or within the individual stages. In these hold zones, the catalyst precursor is maintained at the temperature reached for a particular time, for example from 10 to 120 minutes.

The control of the stage in the temperature range from 80-120° C. to 250-290° C. is particularly important, since the exothermic binder burnout occurs essentially in this temperature range. This stage can, if appropriate, be operated at a lower heating rate, for example from 3 to 10° C. per hour, preferably from 3 to 5° C. per hour, Furthermore, this stage can, if appropriate, comprise a plurality of zones of constant temperature (temperature plateaus). Temperature plateaus are particularly advantageous in the temperature ranges at which thermal decomposition of the binders used occurs.

If appropriate, the introduction of air can be interrupted for a short time during heating-up of the catalyst precursor.

During activation, the amount of air used is advantageously from 0.05 to 5.0 standard m³/h, preferably from 0.05 to 3 standard m³/h and particularly preferably from 0.05 to 1 standard m³/h. As stated above for the heating phase, the air can be diluted with inert gases during activation, too. During activation, which has a duration of at least nine hours, the amount of air can be kept constant increased or reduced. The amount of air is advantageously increased or kept constant during activation. For example, the amount of air can be increased from advantageously 0.05-0.2 standard m³/h to 0.7-1 standard m³/h after from two to four hours. The increase in the amount of air can, if appropriate, also be achieved by dilution with inert gases.

Preactivation is advantageously carried out in an air atmosphere without starting material being fed in.

Preactivation is usually carried out in an inlet gauge range from 0 to 0.45 bar.

Preactivation is advantageously carried out in a fixed-bed reactor which is heated/cooled by means of a salt bath. The fixed-bed reactor advantageously comprises a main reactor comprising a multizone catalyst system and, if appropriate, a downstream finishing reactor. A gas cooler and an apparatus for separating off the product formed are advantageously arranged downstream of the main reactor, or the gas cooler is followed by a finishing reactor, if appropriate a further gas cooler and an apparatus for separating off the product formed. The product formed is, for example, recovered from the reaction gas by desublimation or by means of an appropriate gas scrub. In the preactivation, the air stream is advantageously separated off directly after the main reactor, i.e. before the gas cooler. The separation can be carried out by all means known to those skilled in the art.

As binders, it is possible to use all binders known to those skilled in the art. For example, use is made of copolymers, advantageously in the form of an aqueous dispersion, of vinyl acetate-vinyl laurate, vinyl acetate-acrylate, styrene-acrylate, vinyl acetate-ethylene and acrylic acid-maleic acid, or copolymers of an α-olefin and a vinyl C₂-C₄-carboxylate whose vinyl C₂-C₄-carboxylate content is at least 62 mol %. Preference is given to using copolymers of an α-olefin and a vinyl C₂-C₄-carboxylate whose vinyl C₂-C₄-carboxylate content is at least 62 mol %, as are described in DE-A 103 35 346.

The binders are commercially available as aqueous dispersions having a solids content of, for example, from 35 to 65% by weight. The amount of such binder dispersions used is advantageously from 1 to 30% by weight, based on the amount of suspension used. Preference is given to using from 1 to 20% by weight, in particular from 3 to 12% by weight.

When a small proportion of binder of from about 1 to 5% is used, the amount of air in the second stage in the temperature range from 80-120° C. to 250-290° C. can be reduced to from 0.01 to 2 standard m³/h. Furthermore, the amount of air in the third stage in the temperature range from 250-290° C. to 350-470° C. can be reduced to from 0.05 to 1 standard m³/h. If appropriate, introduction of air can be dispensed with in the temperature range from 250-290° C. to 350-470° C.

When a high proportion of binder of from about 15 to 30% by weight is used, a slow heating rate of from 1 to 5° C. per hour can be selected in a temperature range from 80-120° C. to 250-290° C. Furthermore, the amount of air may, if appropriate, be diluted with inert gases.

The production of the catalyst precursor is known to those skilled in the art and is described, for example, in WO 2005 30380. As catalytically active composition, it is possible to use all compositions known to those skilled in the art, which are described, for example, in WO 2004 103944.

Coating of the catalyst support with the catalytically active composition is usually carried out at coating temperatures of from 75 to 120° C., with coating being able to be carried out under atmospheric pressure or under reduced pressure.

The layer thickness of the catalytically active composition is generally from 0.02 to 0.25 mm, preferably from 0.05 to 0.20 mm. The proportion of active composition in the catalyst is usually from 5 to 25% by weight, mostly from 7 to 15% by weight.

The invention further provides oxidation catalysts produced by the process of the invention. For example, the invention provides oxidation catalysts for preparing carboxylic acids and/or carboxylic anhydrides by catalytic gas-phase oxidation of aromatic hydrocarbons such as benzene, the xylenes, naphthalene, toluene, durene or β-picoline. In this way, it is possible to obtain, for example, benzoic acid, maleic anhydride, phthalic anhydride, isophthalic acid, terephthalic acid, pyromellitic anhydride or niacin.

Furthermore, the process for preparing benzoic acid, maleic anhydride, phthalic anhydride, isophthalic acid, terephthalic acid, pyromellitic anhydride or niacin is generally known.

The preactivation process of the invention differs from the prior art in that precisely defined preactivation steps are adhered to. The preactivation process of the invention enables improved binder burnout and thus optimized start-up behavior to be achieved.

In the case of phthalic anhydride catalysts, the examples show that the catalyst of the invention has the following advantages over the comparative catalyst (cf. Table 2):
a better product quality in respect of the phthalide concentration at a low salt bath temperature,
a better phthalic anhydride (PA) yield and
a shorter running-up time (time until a relatively high o-xylene loading (g/standard m³) is reached).

### EXAMPLES

### A. Production of the Catalysts

### A.1. Production of Catalyst 1

### First Catalyst Zone: Zone 1.1

29.3 g of anatase (BET surface area=7 m²/g), 69.8 g of anatase (BET surface area=20 m²/g), 7.8 g of V₂O₅, 1.9 g of Sb₂O₃, 0.49 g of Cs₂CO₃ were suspended in 550 ml of deionized water and stirred for 18 hours. 50 g of organic binder (i.e. 10% by weight of binder dispersion) comprising a copolymer of vinyl acetate and vinyl laurate (weight ratio=75:25) in the form of a 50% strength aqueous dispersion were added to this suspension. The resulting suspension was subsequently sprayed onto 1200 g of steatite (magnesium silicate) in the form of rings having an external diameter of 7 mm, a length of 7 mm and a wall thickness of 1.5 mm and dried.

An analytical sample showed that the catalytically active composition applied in this way comprised 7.1% by weight of vanadium (calculated as V₂O₅), 1.8% by weight of antimony (calculated as Sb₂O₃) and 0.36% by weight of cesium (calculated as Cs) after calcination at 450° C. for one hour. The BET surface area of the TiO₂ mixture was 15.8 m²/g. The weight of the shell applied was 8% of the total weight of the finished catalyst.

### Second Catalyst Zone: Zone 2.1

24.6 g of anatase (BET surface area=7 m²/g), 74.5 g of anatase (BET surface area=20 m²/g), 7.8 g of V₂O₅, 2.6 g of Sb₂O₃, 0.35 g of Cs₂CO₃ were suspended in 550 ml of deionized water and stirred for 18 hours. 50 g of organic binder (i.e. 10% by weight of binder dispersion) comprising a copolymer of vinyl acetate and vinyl laurate (weight ratio=75:25) in the form of a 50% strength aqueous dispersion were added to this suspension. The resulting suspension was subsequently sprayed onto 1200 g of steatite (magnesium silicate) in the form of rings having an external diameter of 7 mm, a length of 7 mm and a wall thickness of 1.5 mm and dried.

An analytical sample showed that the catalytically active composition applied in this way comprised 7.1% by weight of vanadium (calculated as V₂O₅), 2.4% by weight of antimony (calculated as Sb₂O₃) and 0.26% by weight of cesium (calculated as Cs) after calcination at 450° C. for one hour. The BET surface area of the TiO₂ mixture was 16.4 m²/g. The weight of the shell applied was 8% of the total weight of the finished catalyst.

### Third Catalyst Zone: Zone 3.1

24.8 g of anatase (BET surface area=7 m²/g), 74.5 g of anatase (BET surface area=20 m²/g), 7.8 g of V₂O₅, 2.6 g of Sb₂O₃, 0.13 g of Cs₂CO₃ were suspended in 550 ml of deionized water and stirred for 18 hours. 50 g of organic binder (i.e. 10% by weight of binder dispersion) comprising a copolymer of vinyl acetate and vinyl laurate (weight ratio=75:25) in the form of a 50% strength aqueous dispersion were added to this suspension. The resulting suspension was subsequently sprayed onto 1200 g of steatite (magnesium silicate) in the form of rings having an external diameter of 7 mm, a length of 7 mm and a wall thickness of 1.5 mm and dried.

An analytical sample showed that the catalytically active composition applied in this way comprised 7.1% by weight of vanadium (calculated as V₂O₅), 2.4% by weight of antimony (calculated as Sb₂O₃) and 0.10% by weight of cesium (calculated as Cs) after calcination at 450° C. for one hour. The BET surface area of the TiO₂ mixture was 16.4 m²/g. The weight of the shell applied was 8% of the total weight of the finished catalyst.

### Fourth Catalyst Zone: Zone 4.1

17.2 g of anatase (BET surface area=7 m²/g), 69.1 g of anatase (BET surface area=27 m²/g), 21.9 g of V₂O₅, 1.5 g of NH₄H₂PO₄ were suspended in 550 ml of deionized water and stirred for 18 hours. 55 g of organic binder (i.e. 10% by weight of binder dispersion) comprising a copolymer of vinyl acetate and vinyl laurate (weight ratio=75:25) in the form of a 50% strength aqueous dispersion were added to this suspension. The resulting suspension was subsequently sprayed onto 1200 g of steatite (magnesium silicate) in the form of rings having an external diameter of 7 mm, a length of 7 mm and a wall thickness of 1.5 mm and dried.

An analytical sample showed that the catalytically active composition applied in this way comprised 20.00% by weight of vanadium (calculated as V₂O₅), and 0.38% by weight of phosphorus (calculated as P) after calcination at 450° C. for one hour. The BET surface area of the TiO₂ mixture was 20.9 m²/g. The weight of the shell applied was 8% of the total weight of the finished catalyst.

### A.2. Production of Catalysts 2 and 3

### First Catalyst Zone: Zone 1.2

### Suspension 1:

150 kg of steatite in the form of rings having dimensions of 8 mm×6 mm×5 mm (external diameter×height×internal diameter) were heated in a fluidized-bed apparatus and sprayed with 24 kg of a suspension comprising 155.948 kg of anatase having a BET surface area of 21 m²/g, 13.193 kg of vanadium pentoxide, 35.088 kg of oxalic acid, 5.715 kg of antimony trioxide, 0.933 kg of ammonium hydrogen phosphate, 0.991 g of cesium sulfate, 240.160 kg of water and 49.903 kg of formamide together with 37.5 kg of an organic binder in the form of a 48% strength by weight aqueous dispersion, comprising a copolymer of acrylic acid-maleic acid (weight ratio=75:25) (i.e. 7.5% by weight of binder dispersion).

### Suspension 2:

150 kg of the coated catalyst obtained were heated in a fluidized-bed apparatus and sprayed with 24 kg of a suspension comprising 168.35 kg of anatase having a BET surface area of 21 m²/g, 7.043 kg of vanadium pentoxide, 19.080 kg of oxalic acid, 0.990 g of cesium sulfate, 238.920 kg of water and 66.386 kg of formamide together with 37.5 kg of an organic binder in the form of a 48% strength by weight aqueous dispersion comprising a copolymer of acrylic acid-maleic acid (weight ratio=75:25) (i.e. 7.5% by weight of binder dispersion).

After heat treatment at 450° C. for one hour, an analytical sample showed that the catalytically active composition applied in this way comprised on average 0.08% by weight of phosphorus (calculated as P), 5.75% by weight of vanadium (calculated as V₂O₅), 1.6% by weight of antimony (calculated as Sb₂O₃), 0.4% by weight of cesium (calculated as Cs) and 92.17% by weight of titanium dioxide. The weight of the layers applied was 9.3% of the total weight of the finished catalyst.

### Second Catalyst Zone: Zone 2.2

150 kg of steatite in the form of rings having dimensions of 8 mm×6 mm×5 mm (external diameter×height×internal diameter) were heated in a fluidized-bed apparatus and sprayed with 57 kg of a suspension comprising 140.02 kg of anatase having a BET surface area of 21 m²/g, 11.776 kg of vanadium pentoxide, 31.505 kg of oxalic acid, 5.153 kg of antimony trioxide, 0.868 kg of ammonium hydrogen phosphate, 0.238 g of cesium sulfate, 215.637 kg of water and 44.808 kg of formamide together with 33.75 kg of an organic binder (i.e. 7.5% by weight of binder dispersion) comprising a copolymer of acrylic acid-maleic acid (weight ratio=75:25) until the weight of the applied layer was 10.5% of the total weight of the finished catalyst (analytical sample after heat treatment at 450° C. for one hour). The catalytically active composition applied in this way, i.e. the catalyst coating, comprised on average 0.15% by weight of phosphorus (calculated as P), 7.5% by weight of vanadium (calculated as V₂O₅), 3.2% by weight of antimony (calculated as Sb₂O₃), 0.1% by weight of cesium (calculated as Cs) and 89.05% by weight of titanium dioxide.

### B. Catalyst Bed

### B.1. Catalyst 1

From the bottom upward, 0.70 m of the catalyst zone 4.1, 0.70 m of the catalyst zone 3.1, 0.50 m of the catalyst zone 2.1 and 1.30 m of the catalyst zone 1.1 were introduced into an iron tube having a length of 3.5 m and an internal diameter of 25 mm. The iron tube was surrounded by a salt melt to regulate the temperature; a thermocouple sheath having an external diameter of 4 mm (max. length=2.2 m from the top) with installed withdrawable thermocouple was employed for the catalyst temperature measurement.

### B.2. Catalysts 2 and 3

From the bottom upward, 1.30 m of the catalyst zone 2.2 and 1.50 m of the catalyst zone 1.2 were introduced into an iron tube having a length of 3.5 m and an internal diameter of 25 mm. The iron tube was surrounded by a salt melt to regulate the temperature; a thermocouple sheath having an external diameter of 4 mm (max. length=1.9 m from the top) with installed withdrawable thermocouple was employed for the catalyst temperature measurement.

### C. Preactivation of the Catalysts

Table 1 describes the preactivation according to the invention of the catalysts 1 and 2 and the preactivation of the comparative catalyst 3. The catalysts were heated continuously in the tube reactor, with the amount of air used being changed stepwise. In the preactivation according to the invention, the catalyst 1 was calcined at 400° C. under an amount of air fed in of 0.5 standard m³/h for 24 hours. The catalyst 2 was calcined at 390° C. under an amount of air fed in of 0.1 standard m³/h for 24 hours. The comparative catalyst 3 was calcined at 390° C. under an amount of air fed in of 0.1 standard m³/h for 6 hours.

TABLE 1
Preactivation of the catalysts 1 to 3

| | | Heating | | |
|---|---|---|---|---|
| | Temperature | rate | Hold time | Amount of air |
| 1st | Room tempera- | 8° C./h | — | 0.5 standard |
| stage | ture to 100° C. | | | m³/h of air |
| 2nd | 100 to 270° C. | 8° C./h | — | 3.0 standard |
| stage | | | | m³/h of air |
| 3rd | 270 to 390° C. | 8° C./h | 24 h at 400° C. | 0.5 standard |
| stage | | | [catalyst 1] | m³/h of air |
| | | | 24 h at 390° C. | 0.1 standard |
| | | | [catalyst 2] | m³/h of air |
| | | | 6 h at 390° C. | 0.1 standard |
| | | | [catalyst 3] | m³/h of air |
| | | | (comparative | |
| | | | example) | |

### D. Oxidation of o-Xylene to PA

### D.1 Model Tube Test of the Catalysts

4.0 standard m³/h of air having a loading of 99.2% strength by weight o-xylene of from 0 to 100 g/standard m³ were passed through the reactor tube from the bottom upward. At 45-70 g of o-xylene/standard m³, the results summarized in Table 2 were obtained (“PA yield” refers to the amount of phthalic anhydride obtained in percent by weight, based on 100% pure o-xylene).

TABLE 2
Preparation of PA at an o-xylene loading of 45-70 g/
standard m³ in 4.0 standard m³/h of air using a
2- and 4-zoned catalyst (PA yield is an average PA yield).

| | | | Catalyst 3 |
|---|---|---|---|
| | | | comparative |
| Model tube results | Catalyst 1 | Catalyst 2 | example |
| o-Xylene loading | 70 | 63 | 45 |
| [g/standard m³] | | | |
| Salt bath temperature | 365 | 359 | 375 |
| [° C.] | | | |
| Running time | 12 | 12 | 12 |
| [days] | | | |
| PA yield | 114.1 | 113.4 | 111.1 |
| [m/m-%] | | | |
| Phthalide | 0.11 | 0.07 | 0.16 |
| [% by weight] | | | |

1-10. (canceled)

11. A process comprising: providing a catalyst precursor; and heating the catalyst precursor to a temperature of at least 350° C. in an atmosphere comprising air, wherein air is fed into the atmosphere at a rate of 0.05 to 4.0 standard m³/h, and wherein the catalyst precursor is activated at a temperature of at least 350° C. for more than 9 hours.

12. The process according to claim 11, wherein the catalyst precursor is activated at a temperature of at least 350° C. for at least 12 hours.

13. The process according to claim 11, wherein the catalyst precursor is heated to a temperature of at least 370° C. and is activated at least 370° C.

14. The process according to claim 11, wherein the catalyst precursor is heated at a heating rate of 3 to 12° C./h.

15. The process according to claim 11, wherein air is fed into the atmosphere during the activation at a rate of 0.05 to 3 standard m³/h.

16. The process according to claim 11, wherein the activation is carried out in a fixed-bed reactor heated by a salt bath.

17. The process according to claim 11, wherein heating of the catalyst precursor to a temperature of at least 350° C. comprises a first heating stage wherein the catalyst precursor is heated from room temperature to 80-120° C. using an amount of air of from 0.05 to 3 standard m³/h; a second heating stage wherein the catalyst precursor is heated from 80-120° C. to 250-290° C. using an amount of air of from 1 to 4.5 standard m³/h; and a third heating stage wherein the catalyst precursor is heated from 250-290° C. to 350-470° C. using an amount of air of from 0.05 to 2.5 standard m³/h.

18. The process according to claim 11, wherein the activation is carried out in a fixed-bed reactor comprising a multizone main reactor, and an optional finishing reactor wherein the air stream is separated off after the main reactor.

19. An oxidation catalyst prepared by the process according to claim 11.

20. A process comprising providing an oxidation reactant, oxidizing the reactant in the presence of the oxidation catalyst according to claim 19 to form an oxidized product.

21. The process according to claim 20, wherein the oxidized product comprises a compound selected from the group consisting of benzoic acid, maleic anhydride, phthalic anhydride, isophthalic acid, terephthalic acid, pyromellitic anhydride, niacin and combinations thereof.
